(19) United States
(12) Patent Application Publication
(10) Pub. No.: US 2014/0228631 A1
(43) Pub. Date: Aug. 14, 2014

(54) SURGICAL ROBOT AND CONTROL METHOD FOR THE SAME

(71) Applicant: SAMSUNG ELECTRONICS CO., LTD., Suwon-si (KR)

(72) Inventors: No San Kwak, Suwon-si (KR); Hee Kuk Lee, Suwon-si (KR); Woong Kwon, Seongnam-si (KR)

(73) Assignee: SAMSUNG ELECTRONICS CO., LTD., Suwon-si (KR)

(21) Appl. No.: 13/941,934

(22) Filed: Jul. 15, 2013

(30) Foreign Application Priority Data
Feb. 14, 2013 (KR) 10-2013-0015788

(51) Int. Cl. A61B 19/00; A61B 1/04; A61B 1/00; B25J 9/16

(52) U.S. Cl. CPC A61B 19/2203; B25J 9/1697; A61B 19/5212; A61B 1/04; A61B 1/00149; A61B 19/5225; Y10S 901/47
USPC 600/102; 700/259; 606/130

(57) ABSTRACT

A surgical robot and a control method for the enables measurement of external force applied to a surgical tool of a slave device. In the surgical robot, the slave device may include an external force measurement unit, an image capture unit, and a controller. The external force measurement unit may include a force sensor attached to a surgical instrument to measure external force applied to a surgical tool provided at the surgical instrument, and a visual information display unit connected to the force sensor to display visual information corresponding to the external force output from the force sensor. The image capture unit acquires an image with regard to the visual information. The controller extracts the visual information from the image using image processing, and converts the extracted visual information into corresponding external force information.

### CROSS-REFERENCE TO RELATED APPLICATIONS

This application claims the priority benefit of Korean Patent Application No. 10-2013-0015788, filed on Feb. 14, 2013 in the Korean Intellectual Property Office, the disclosure of which is incorporated herein by reference.

### BACKGROUND

1. Field

Embodiments relate to a surgical robot and a control method for the same, which enable measurement of external force applied to a surgical tool of a slave device.

2. Description of the Related Art

Minimally invasive surgery refers to surgical methods less invasive than open surgeries. For example, laparotomy (a type of open surgery) uses a relatively large surgical incisions through a part of a human body (e.g., the abdomen). However, in minimally invasive surgery, after forming at least one small port of 0.5 cm˜1.5 cm (incisions or invasive holes) through the abdominal wall, an operator inserts an endoscope and various surgical tools through the port, to perform surgery while viewing images.

As compared to laparotomy, minimally invasive surgery has several advantages, such as low pain after surgery, early recovery, early restoration of ability to eat, short hospitalization, rapid return to daily life, and superior cosmetic effects owing to a small incision part. Accordingly, minimally invasive surgery has been used in gall resection, prostate cancer, and herniotomy operations, etc, and the use range thereof increasingly expands.

A surgical robot for use in minimally invasive surgery includes a master device and a slave device. The master device generates a manipulation signal entered by a doctor to transmit the control signal to the slave device. The slave device directly performs manipulation required for surgery of a patient upon receiving the control signal from the master device. The master device and the slave device may be integrated with each other, or may be separately arranged in an operating room.

The slave device includes at least one robot arm. A surgical instrument is mounted to an end of each robot arm, and in turn a surgical tool is mounted to an end of the surgical instrument.

In minimally invasive surgery using the aforementioned surgical robot, the surgical tool of the slave device and the surgical instrument, to which the surgical tool is mounted, are introduced into the body of a patient to perform required procedures. After the surgical tool and the surgical instrument enter the human body, an internal situation is visible from images collected using the surgical tool such as an endoscope.

### SUMMARY

In an aspect of one or more embodiments, there is provided a surgical robot and a control method for the same, which enable accurate measurement of external force applied to a surgical tool without loss.

In an aspect of one or more embodiments, there is provided a surgical robot which includes a slave device that includes a robot arm, to which a surgical instrument provided with a surgical tool is coupled, and a master device that controls operation of the slave device, wherein the slave device includes an external force measurement unit that includes a force sensor attached to the surgical instrument to measure external force applied to the surgical tool, and a visual information display unit connected to the force sensor to receive the measured external force from the force sensor and display visual information corresponding to the received external force, an image capture unit that acquires an image with regard to the visual information displayed on the visual information display unit, and a controller that extracts the visual information from the acquired image from the image capture unit using image processing, and converts the extracted visual information into corresponding external force information.

In an aspect of one or more embodiments, there is provided a control method for a surgical robot including a slave device that includes a robot arm, to which a surgical instrument provided with a surgical tool is coupled, and a master device that controls operation of the slave device, includes measuring external force applied to the surgical tool, displaying visual information corresponding to the measured external force, acquiring an image with regard to the displayed visual information, and calculating external force information corresponding to the visual information using the acquired image.

According to another aspect of one or more embodiments, there is provided at least one non-transitory computer readable medium storing computer readable instructions to implement methods of one or more embodiments.

### BRIEF DESCRIPTION OF THE DRAWINGS

These and/or other aspects of embodiments will become apparent and more readily appreciated from the following description of embodiments, taken in conjunction with the accompanying drawings of which:

FIG. 1 is a plan view showing an overall configuration of a surgical robot of an embodiment;

FIG. 2 is a block diagram showing a configuration for measurement of external force applied to a surgical tool of a slave device included in the surgical robot of an embodiment;

FIG. 3 is a block diagram showing a configuration of a visual information display unit of FIG. 2;

FIG. 4 is a block diagram showing one embodiment of the visual information display unit of FIG. 3;

FIG. 5 is a block diagram showing another embodiment of the visual information display unit of FIG. 3;

FIG. 6 is a view showing one embodiment in which external force is visibly represented and recognized via the visual information display unit of FIG. 4;

FIG. 7 is a view showing another embodiment in which external force is visibly represented and recognized via the visual information display unit of FIG. 5;

FIG. 8 is a flowchart showing a control method for the surgical robot with regard to operation of the salve device of an embodiment;

FIG. 9 is a flowchart showing one detailed example of Operation S830 of FIG. 8; and

FIG. 10 is a flowchart showing another detailed example of Operation S830 of FIG. 8.

### DETAILED DESCRIPTION

In the following description of embodiments, a detailed description of known functions and configurations incorporated herein will be omitted when it may make the subject matter of embodiments rather unclear. Herein, the terms first, second, etc. are used simply to discriminate any one element from other elements, and the elements should not be limited by these terms.

Reference will now be made in detail to embodiments, examples of which are illustrated in the accompanying drawings, wherein like reference numerals refer to the like elements throughout. Embodiments are described below to explain the present disclosure by referring to the figures.

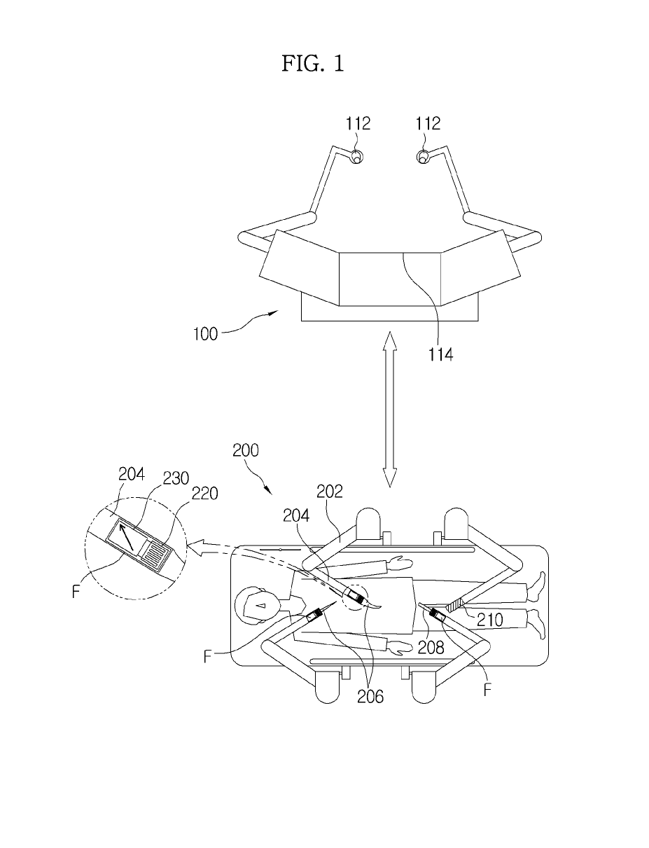

FIG. 1 is a plan view showing an overall configuration of a surgical robot of an embodiment.

Referring to FIG. 1, the surgical robot may include a slave device 200 to perform surgery on a patient who lies on an operating table, and a master device 100 to assist an operator (e.g., a doctor) in remotely controlling the slave device 200.

Although the master device 100 and the slave device 200 may be physically separate components as shown in FIG. 1, embodiments are not limited thereto. In one example, the master device 100 and the slave device 200 may be integrated with each other.

As exemplarily shown in FIG. 1, the master device 100 may include an input unit 112 and a display unit 114.

The input unit 112 may receive an instruction for selection of an operation mode of the surgical robot, or an instruction for remote control of operations of the slave device 200 input by the operator. In an embodiment, the input unit 112 may include any one selected from among a haptic device, a clutch pedal, a switch, and a button, but is not limited thereto. In one example, a voice recognition device may be used.

In FIG. 1, a haptic device is exemplarily shown as one example of the input unit 112. Although FIG. 1 shows an input unit 112 includes two handles, this is given by way of example and embodiments are not limited thereto. For example, the input unit 112 may include one handle, or three or more handles.

The master device 100 may generate a control signal corresponding to operator manipulation of the handle, and transmit the control signal to the slave device 200. Operations of the slave device 200 may be controlled based on the transmitted control signal.

The display unit 114 of the master device 100 may display an image input by an endoscope 210 of the slave device 200.

The display unit 114 may include one or more monitors such that the respective monitors individually display data required for surgery. For example, if the display unit 114 includes three monitors, one of the monitors may display an image input via the endoscope 210, i.e. an image of a surgical region inside the body of a patient, and the other two monitors may respectively display data regarding (related to, corresponding to, or pertaining to) an operating state of the slave device 200 and data regarding (related to, corresponding to, or pertaining to) the patient. The number of monitors may be determined in various ways according to the type or kind of data to be displayed.

The master device 100 and the slave device 200 may construct a network. A network may be a wired network, a wireless network, or a combination thereof.

The master device 100, connected to the slave device 200 via the network, may transmit a control signal to the slave device 200. The “control signal” may include a control signal for position adjustment and operation of surgical tools 206 and 208 coupled to surgical instruments 204 of the slave device 200 and a control signal for position adjustment of the endoscope 210 coupled to the surgical instrument 204, but embodiments are not limited thereto. If it is necessary to transmit the respective control signals for the surgical tools 206 and 208 and the endoscope 210 simultaneously or at similar times, the respective control signals may be transmitted independently of each other.

The term “independent” transmission of the respective control signals may refer to no interference between the control signals, and also may refer to any one control signal that has no effect on the other control signal. To ensure independent transmission of the plurality of control signals, various methods, for example, transmission of additional header data regarding the respective control signals, transmission of the respective control signals based on a generation sequence thereof, or transmission of the control signals based on a preset order of priority, may be used.

In addition, the slave device 200, connected to the master device 100 via the network, may feed back, e.g., data regarding external force applied to the surgical tool and data regarding an image input by the endoscope 210, to the master device 100.

The slave device 200 may include a plurality of robot arms 202, the surgical instruments 204 mounted to ends of the robot arms 202, a variety of surgical tools 206 and 208 mounted to ends of the surgical instruments 204, and the endoscope 210. Although not shown in FIG. 1, the slave device 200 may include a body (not shown) to which the plurality of robot arms 202 is coupled. The body (not shown) may support the plurality of robot arms 202.

In addition, although not shown in detail in FIG. 1, each of the plurality of robot arms 202 may include a plurality of links and a plurality of joints. Each joint serves to connect two links to each other, and may have one (1) degree of freedom (DOF) or more than one degree of freedom.

The “DOF” refers to a DOF with regard to kinematics or inverse kinematics, i.e. the DOF of a mechanism.

The “DOF of a mechanism” refers to the number of independent motions of a mechanism, or the number of variables that determine independent motions at relative positions between links. For example, an object in a 3D space defined by X-, Y-, and Z-axes has one or more of 3 DOF to determine a spatial position of the object (a position on each axis) and 3 DOF to determine a spatial orientation of the object (a rotation angle relative to each axis).

More specifically, it will be appreciated that if an object is movable along each of X-, Y- and Z-axes and is rotatable about each of X-, Y- and Z-axes, it will be appreciated that the object has 6 DOF. To this end, each joint of the robot arm 202 may be provided with a drive unit (not shown) that is driven in response to the control signal of the master device 100.

For example, if the control signal is transmitted from the master device 100 to the slave device 200 when the operator manipulates the input unit 112 of the master device 100, the slave device 200 may drive the drive unit (not shown) using the transmitted control signal to control movement of each joint of the robot arm 202.

Although each joint of the robot arm 202 of the slave device 200 may be configured to move in response to the control signal of the master device 100, the joint may be moved by external force as well. That is, an assistant who is located near an operating table may manually move each joint of the robot arm 202.

Although not shown in detail in FIG. 1, in one example, the surgical instrument 204 may include a housing mounted to the end of the robot arm 202, and a shaft extending from the housing by a predetermined length.

A drive wheel (not shown) may be coupled to the housing. The drive wheel (not shown) may be connected to the surgical tool 206 or 208 via, e.g., a wire, so as to be operated to follow the surgical tool 206 or 208 via rotation of the drive wheel (not shown). To this end, an actuator to rotate the drive wheel (not shown) may be installed to the end of the robot arm 202. Of course, the operating mechanism of the surgical tools 206 and 208 is not necessarily constructed as described above, and various other electrical/mechanical mechanisms to realize required motions for the surgical tools 206 and 208 may be applied.

The variety of surgical tools 206 and 208 may include a skin holder, a suction line, a knife, scissors, a grasper, a needle holder, a staple applier, a scalpel, etc., but are not in any way limited thereto. Any other known tools required for surgery may be used.

In general, surgical tools may be basically classified into a main surgical tool and an auxiliary surgical tool. The “main surgical tool” may refer to a tool that performs direct surgical motions, such as, e.g., cutting and suturing on a surgical region (e.g., a knife or a surgical needle). The “auxiliary surgical tool” may refer to a tool that does not perform direct motions on a surgical region and assists motion of the main surgical tool (e.g., a skin holder).

Likewise, the endoscope 210 does not perform direct motions on a surgical region and is used to assist a motion of the main surgical tool. Therefore, the endoscope 210 may be considered as corresponding to the auxiliary surgical tool in a broad sense. The endoscope 210 may be selected from among various surgical endoscopes, such as a thoracoscope, an arthroscope, and a rhinoscope, in addition to a celioscope that is mainly used in robotic surgery.

Although not shown in FIG. 1, the slave device 200 may further include a monitor (not shown) that may display an image regarding a surgical region inside the body of a patient input by the endoscope 210.

Although FIG. 1 shows the slave device 200 having multiple ports, multiple robot arms, multiple surgical instruments, and multiple surgical tools, this is given by way of example, and additionally, embodiments may be applied to a surgical robot system including a slave device having a single-port, multiple robot arms, multiple surgical instruments, and multiple surgical tools, a slave device having a single port, a single robot arm, multiple surgical instruments, multiple surgical tools, and various other slave devices.

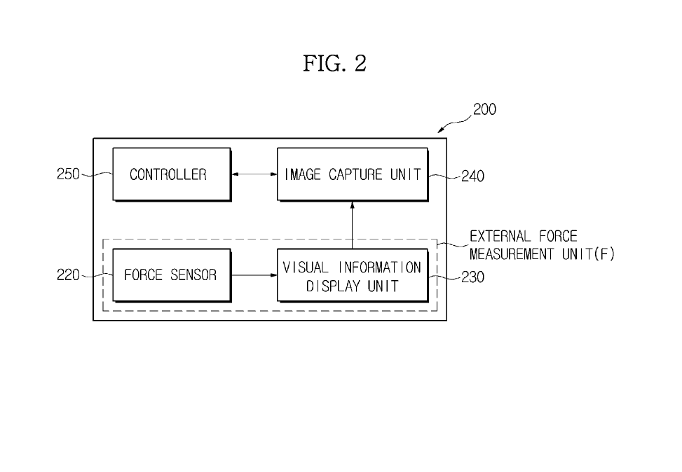

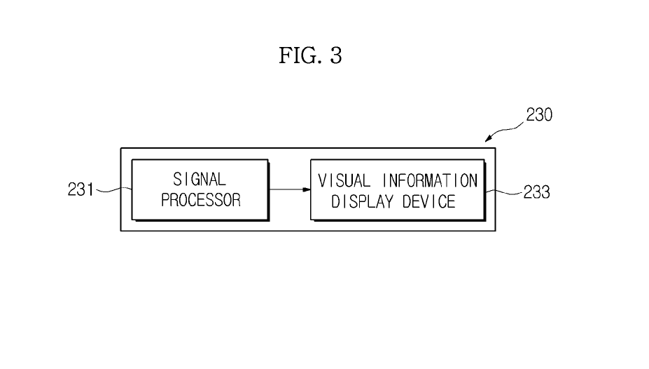

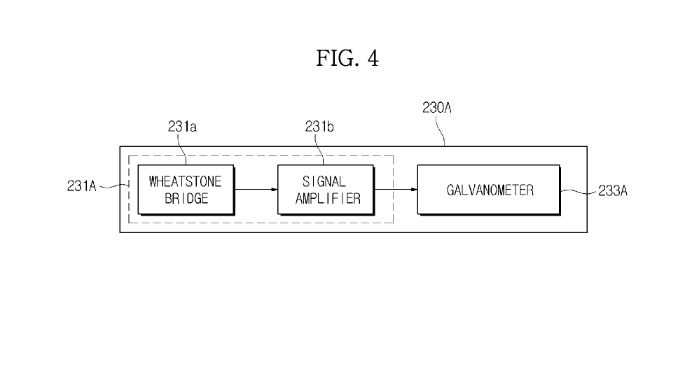

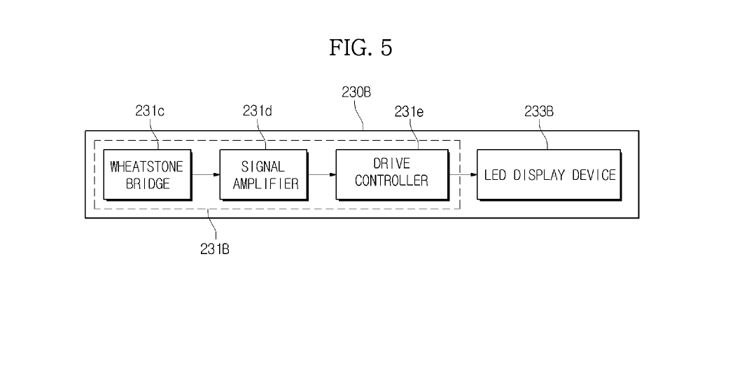

FIG. 2 is a block diagram showing a configuration for measurement of external force applied to the surgical tool of the slave device included in the surgical robot in an embodiment, FIG. 3 is a block diagram showing a configuration of a visual information display unit of FIG. 2, FIG. 4 is a block diagram showing an embodiment of the visual information display unit of FIG. 3, and FIG. 5 is a block diagram showing another embodiment of the visual information display unit of FIG. 3.

Referring to FIG. 2, the configuration for measurement of external force applied to the surgical tool of the slave device 200 may include an external force measurement unit F, which includes a force sensor 220 attached to the end of the surgical instrument 204, to which the surgical tool is mounted, to measure external force applied to the surgical tool and a visual information display unit 230 connected to the force sensor 220 to receive the measured external force from the force sensor 220 and display visual information corresponding to the transmitted external force. The configuration for measurement of external force may further include an image capture unit 240 which acquires an image with regard to (pertaining to, corresponding to, or related to) visual information displayed on the visual information display unit 230, and a controller 250 which receives the image with regard to (pertaining to, corresponding to, or related to) the visual information acquired from the image capture unit 240, extracts visual information within the image using image processing, and converts the extracted visual information into corresponding external force information.

The force sensor 220 is a sensor that serves to detect force. The force sensor 220 may be classified, based on conversion from force into electric current, into a force sensor that utilizes deformation of an elastic element as a primary conversion factor, and a force sensor that utilizes equilibrium between the measured force and preset force. the force sensor utilizing deformation of an elastic element may include one that detects a deformation degree, one that utilizes physical effects due to deformation, and one that utilizes variation in the rate of vibration due to deformation, for example.

Although an embodiment may utilize a strain gauge as the force sensor 220, the force sensor is not in any way limited thereto, and all force sensors known in the art may be applied.

The strain gauge is a sensor to measure deformation of an object caused by external force. To this end, the strain gauge may be attached to the object to be measured so as to judge whether or not deformation occurs and to measure a deformation degree. For example, the strain gauge is fabricated by forming a latticed resistor wire or a resistor foil acquired via a photo etching process on an electrically insulated thin base and attaching a lead cable to the resistor wire or the resistor foil. The strain gauge is based on characteristics of a metal, resistance of which varies according to variation in the length thereof.

Specifically, if tensile force is applied to the strain gauge, a length of the resistor wire formed on the base of the strain gauge is increased, and resistance is increased in proportion thereto. Conversely, if stress is applied to the strain gauge, the length of the resistor wire formed on the base of the strain gauge is reduced, and resistance is reduced in proportion thereto. As such, whether or not the object is deformed may be judged and the magnitude of the applied external force may be calculated by measuring the generated resistance.

Surgical robots according to the related art frequently use the strain gauge to measure external force applied to surgical tools. However, in the related art, it may be necessary to connect an electric wire of the strain gauge to a controller, which may require an increase in the length of the electric wire. Moreover, the increased length of the electric wire may increase noise, causing greater signal loss from the strain gauge to the controller.

For this reason, in an embodiment, as exemplarily shown in FIG. 2, an electric wire connected to the force sensor 220 may be connected to the visual information display unit 230 adjacent to the force sensor 220, rather than being directly connected to the controller 250. Hereinafter, although the strain gauge will be described as a specific example of the force sensor 220, the force sensor 220 that may be used as described above is not limited to the strain gauge.

More specifically, in an embodiment, the electric wire connected to the strain gauge 220 is not connected to the controller 250 and thus a resistance output from the strain gauge 220 is not input to the controller 250. Instead, the electric wire is connected to the visual information display unit 230 located close to the strain gauge 220 such that resistance output from the strain gauge 220 in response to external force applied to the surgical tool is displayed as corresponding visual information.

The visual information display unit 230 serves to visually represent the resistance output from the strain gauge 220, i.e. an electric signal. As exemplarily shown in FIG. 3, the visual information display unit 230 may basically include a visual information display device 233 that displays the resistance as visual information, and a signal processor 231 that converts the resistance output from the strain gauge 220 into a signal compatible with the visual information display device 233 and transmits the converted signal to the visual information display device 233.

Although FIGS. 4 and 5 show two detailed examples of the visual information display unit 230 according to an embodiment, this is given by way of example and embodiments are not limited thereto. Hereinafter, the visual information display units exemplarily shown in FIGS. 4 and 5 respectively are designated by different reference numerals 230A and 230B.

First, FIG. 4 is a view showing a galvanometer 233A as one example of the visual information display device.

Referring to FIG. 4, a visual information display unit 230A may include a signal processor 231A, which includes a Wheatstone bridge 231a that converts the resistance output from the strain gauge 220 into a voltage to output the voltage and a signal amplifier 231b that amplifies the voltage output from the Wheatstone bridge 231a. The visual information display unit 230A may further include a galvanometer 233A which measures the amplified voltage output from the signal processor 231A to display the measured voltage as visual information.

The galvanometer 233A is an instrument that measures extremely low levels of current, voltage, and electricity charge of an electric circuit. The galvanometer 233A may be referred to as a current indicator. Although a current meter is used when measuring relatively high levels of current, generally, a current indicator is used when measuring relatively low levels of current.

A current indicator may be classified into a Direct Current (DC) indicator and an Alternating Current (AC) indicator. The DC indicator is configured such that a movable coil is connected between poles of a strong magnet, and measures the presence/absence of current as the movable coil is tilted by force applied thereto when a low level of current is applied to the coil. Therefore, the DC indicator is also referred to as a ‘movable coil type current indicator’. A representative example is a pointer type current indicator that is relatively simply to use. The ‘pointer type current indicator’ may detect current from movement of a pointer mounted to the movable coil.

Accordingly, in an embodiment, the electric wire connected to the strain gauge 220 is connected to the galvanometer 233A, such that the resistance output from the strain gauge 220 is represented as pointer movement. A ‘pointer movement’ may represent visual information.

In general, to measure delicate variation in the resistance output from the strain gauge 220, it may be necessary to connect the Wheatstone bridge 231a having a voltage drive source to the strain gauge 220. This is because a voltage signal may be processed. The Wheatstone bridge 231a is a circuit including a plurality of resistors connected to the strain gauge 220 in series such that variation in the resistance output from the strain gauge 220 causes variation in output voltage. Consequently, the voltage output from the Wheatstone bridge 231a serves as an output signal from the strain gauge 220.

The voltage output from the Wheatstone bridge 231a is extremely low, and therefore is typically amplified to 1000˜10000 times via the signal amplifier 231b for accurate measurement. The galvanometer 233A is connected to an output terminal of the signal amplifier 231b and serves to measure the amplified voltage output from the signal amplifier 231b and to move and display a pointer based on the measured magnitude of voltage. The Wheatstone bridge 231a, the signal amplifier 231b, and the galvanometer 233A may be physically separated from each other, or may be integrated with each other.

Next, FIG. 5 is a view showing a Light Emitting Diode (LED) display device as another example of the visual information display device.

Referring to FIG. 5, a visual information display unit 230B includes a signal processor 231B, and an LED display device 233B that is operated in response to a drive control signal output from the signal processor 231B to display visual information. The signal processor 231B includes a Wheatstone bridge 231c that converts the resistance output from the strain gauge 220 into a voltage and outputs the same, a signal amplifier 231d that amplifies the voltage output from the Wheatstone bridge 231c, and a drive controller 231e that receives the amplified voltage from the signal amplifier 231d, calculates external force corresponding to the received voltage, and outputs a drive control signal for display of visual information corresponding to the calculated external force to the LED display device 233B so as to control driving of the LED display device 233B.

The LED display device 233B is commonly used for outdoor billboards, vehicular rear sign-boards, guide panels inside subway terminals, etc. The LED display device 233B generates light by applying voltage to an LED matrix in which a plurality of LEDs is arrayed, thereby displaying graphic data, such as letters and pictures.

An LED is a semiconductor device that emits light by applying current to a compound, such as gallium-arsenide, etc. The LED is configured such that electrons and positively charged atoms referred to as holes recombine at the center of electrodes attached to upper and lower sides of a conductive material. When current passes through the conductive material, the energy of recombination is released as photons of light. Characteristics of the conductive material determine the color of the light that is emitted.

As a plurality of LEDs that is operated upon receiving current is arrayed in a matrix form and current is selectively supplied or not supplied to each of the LEDs, desired graphic data may be displayed by driving specific LEDs.

Although not shown in detail in FIG. 5, the drive controller 231e may include a calculator (not shown) to calculate external force corresponding to the input voltage. Although the calculator (not shown) may calculate the corresponding external force by substituting the voltage into a preset function, this is given by way of example, and a configuration and method for calculation of external force are not limited thereto.

The visual information display unit 230B may include a storage unit (not shown) in which a drive control signal with regard to visual information corresponding to the calculated external force is stored. The drive controller 231e may first calculate external force, and thereafter read the drive control signal for display of visual information corresponding to the calculated external force from the storage unit (not shown) to output the drive control signal to the LED display device 233B.

The ‘drive control signal’ may be a signal to control whether or not current is applied to the respective LEDs included in the LED display device 233B, but is not in any way limited thereto, and may include all known control signals to drive the LED display device 233B.

That is, in an embodiment, the resistance output from the strain gauge 220, i.e. an invisible electric signal is represented as visual information. The galvanometer 233A as exemplarily shown in FIG. 4 visually represents the resistance output from the strain gauge 220 via movement of the pointer, whereas the LED display device 233B as exemplarily shown in FIG. 5 may visually represent letters including numbers or specific figures.

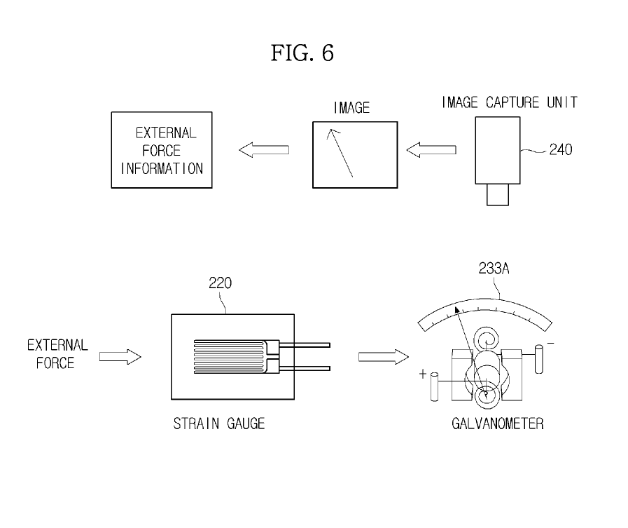

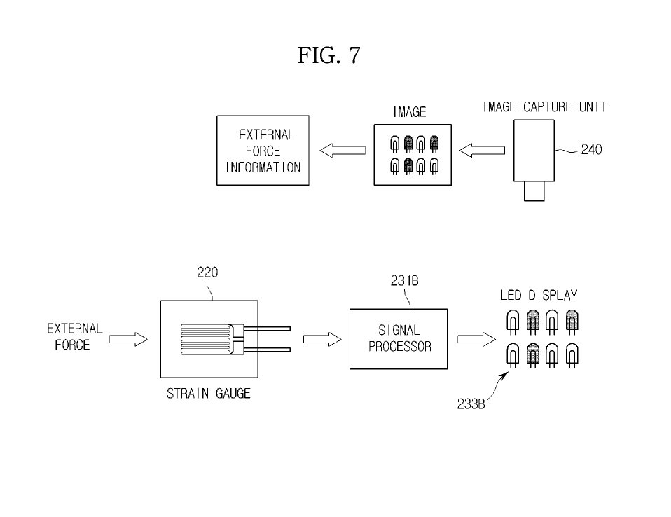

Embodiments for visible representation and recognition of external force using the visual information display units of FIGS. 4 and 5 are respectively shown in FIGS. 6 and 7. These embodiments are given by way of example, and the disclosure is not in any way limited thereto.

Referring to FIG. 6, as an output terminal of the strain gauge 220 is electrically connected to the galvanometer 233A, the galvanometer 233A measures the resistance output from the strain gauge 220, and displays the measured value by moving the pointer according to the measured resistance.

Referring to FIG. 7, as the output terminal of the strain gauge 220 is connected to the signal processor 231B and an output terminal of the signal processor 231B is connected to the LED display device 233B, the signal processor 231B converts the resistance output from the strain gauge 220 in to a corresponding voltage and amplifies the converted voltage. Then, the signal processor 231B calculates external force corresponding to the amplified voltage, and outputs a drive control signal corresponding to the calculated external force to display the visual information on the LED display device 233B.

As such, an image with regard to the visual information displayed via the galvanometer 233A or the LED display device 233B may be acquired using the image capture unit 240 as exemplarily shown in FIGS. 6 and 7. The image capture unit 240 may include a Charge Coupled Device (CCD) camera and an endoscope camera, but is not in any way limited thereto, and any other device may be used so long as it may form an image. If the image capture unit 240 includes a separate camera, rather than the endoscope mounted in the surgical robot, an additional robot arm 202 for coupling of the image capture unit 240 may be provided.

After acquiring the image with regard to the visual information displayed via the galvanometer 233A or the LED display device 233B, the image capture unit 240 transmits the acquired image to the controller 250. The controller 250 may perform any of various known image processing methods on the image transmitted from the image capture unit 240, thereby serving to extract visual information from the image and to calculate external force information corresponding to the extracted visual information.

For example, the controller 250 may extract visual information, such as the direction of the pointer of the galvanometer 233A, a number of a scale indicated by the pointer, etc. Alternatively, the controller 250 may extract visual information, such as letters, figures, pictures, etc., displayed on the LED display device 233B, and calculate external force information corresponding to the visual information.

the ‘image processing method’ may be freely selected from among known methods without limitation. In addition, it is clear that known various improved methods (e.g., filters) may be used to eliminate errors and improve accuracy of feature extraction.

The slave device 200 may further include a storage unit (not shown) in which the external force information matched to the visual information extracted from the image is stored, and a communication unit (not shown). The controller 250 may read the external force information corresponding to the extracted visual information from the storage unit (not shown) after image processing, and then may transmit the read external force information to the master device 100 using the communication unit (not shown). In addition, the master device 100 may feed back the external force information to the input unit 112 to allow the operator to sense the external force applied to the surgical tools 206 and 208.

Although an embodiment in which the image with regard to the visual information acquired by the image capture unit 240 is transmitted to the controller 250 of the slave device 200 has been described above, this is given by way of example, and the image with regard to the visual information acquired by the image capture unit 240 may be transmitted to the controller (not shown) of the master device 100 via a network. The controller (not shown) of the master device 100 may extract visual information by performing image processing on the visual information transmitted from the image capture unit 240, and convert the extracted visual information into corresponding external force information and feed back the converted information to the input unit 112.

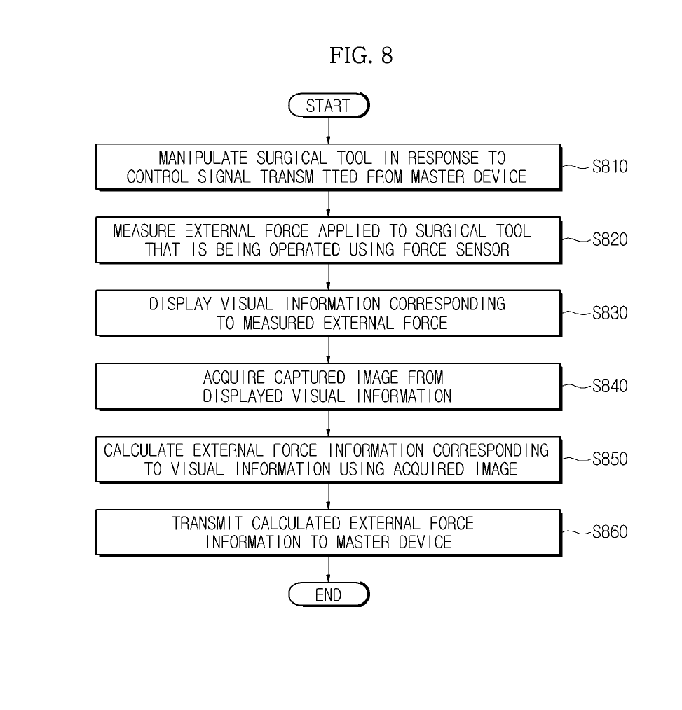

FIG. 8 is a flowchart showing a control method for the surgical robot with regard to sequential operations of the salve device in an embodiment. Hereinafter, the control method for the surgical robot will be described with reference to the configuration as exemplarily shown in FIGS. 1 to 5.

First, the slave device 200 operates the surgical tools 206 and 208 in response to control signals transmitted from the master device 100 (S810).

The ‘control signals’ may be generated as the operator (e.g., doctor) manipulates the input unit 112 of the master device 100. The “control signals” may include a control signal for position adjustment and operation of the surgical tools 206 and 208 coupled to the surgical instruments 204 of the slave device 200 and a control signal for position adjustment of the endoscope 210 coupled to the surgical instrument 204, but are not in any way limited thereto. If it is necessary to transmit the respective control signals for the surgical tools 206 and 208 and the endoscope 210 simultaneously or at similar times, the respective control signals may be transmitted independently of each other.

An “independent” transmission of the respective control signals may refer to no interference between the control signals, and also refer to any one control signal that has no effect on the other control signal. To ensure independent transmission of the plurality of control signals, various methods, for example, transmission of additional header data regarding the respective control signals, transmission of the respective control signals based on a generation sequence thereof, or transmission of the control signals based on a preset order of priority, may be used.

Next, external force applied to the surgical tools 206 and 208 that are being operated in response to the transmitted control signals is measured (S820).

Measurement of the external force applied to the surgical tools 206 and 208 may be performed using the force sensor 220 attached to the end of the surgical instrument 204 provided with the surgical tool 206 or 208 as exemplarily shown in FIG. 1. Although the force sensor 220 may include a strain gauge in an embodiment, the force sensor is not in any way limited thereto, and all force sensors known in the art may be used.

The strain gauge is a sensor to measure deformation of an object caused by external force. To this end, the strain gauge may be attached to the object to be measured so as to judge whether or not deformation occurs and to measure a deformation degree. For example, the strain gauge is fabricated by forming a latticed resistor wire or a resistor foil acquired via a photo etching process on an electrically insulated thin base and attaching a lead cable to the resistor wire or the resistor foil. The strain gauge is based on characteristics of a metal, resistance of which varies according to variation in the length thereof.

Specifically, if tensile force is applied to the strain gauge, a length of the resistor wire formed on the base of the strain gauge is increased, and resistance is increased in proportion thereto. Conversely, if stress is applied to the strain gauge, the length of the resistor wire formed on the base of the strain gauge is reduced, and resistance is reduced in proportion thereto. As such, whether or not the object is deformed may be judged and the magnitude of the applied external force may be calculated by measuring the generated resistance.

Using the strain gauge as the force sensor 220 in the following operations will be described by way of example, but the force sensor 220 is not limited to the strain gauge as described above.

Next, visual information corresponding to the measured external force is displayed (S830).

Various methods of displaying visual information corresponding to the measured external force may be present. Hereinafter, although a visual information display method using the galvanometer 233A and the LED display device 233B will be described, this is given by way of example and the visual information display device is not limited thereto. Any other device may be used so long as it may measures an electric signal and visually display the signal.

First, the visual information display method using the galvanometer 233A will be described in detail.

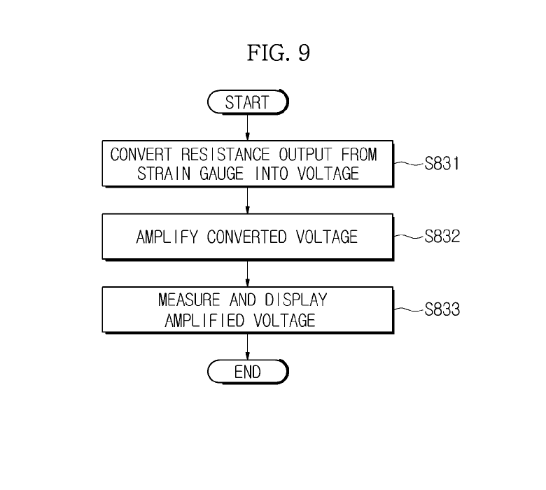

As exemplarily shown in FIG. 9, the resistance output from the strain gauge 220 is converted into a voltage signal (S831). The converted voltage is amplified for easy measurement (S832). The galvanometer 233A measures the amplified voltage, and moves the pointer to indicate the measured magnitude of voltage (S833).

A conversion of the resistance output from the strain gauge 220 into the voltage may be performed using the Wheatstone bridge 231a. The Wheatstone bridge 231a is a circuit including a plurality of resistors connected to the strain gauge 220 in series such that variation in the resistance output from the strain gauge 220 causes variation in output voltage. Consequently, the voltage output from the Wheatstone bridge 231a serves as an output signal from the strain gauge 220.

The voltage output from the Wheatstone bridge 231a may be extremely low, and therefore is typically amplified to 1000˜10000 times via the signal amplifier 231b for accurate measurement. The galvanometer 233A is connected to an output terminal of the signal amplifier 231b and serves to measure the amplified voltage output from the signal amplifier 231b and to move and display a pointer based on the measured magnitude of voltage.

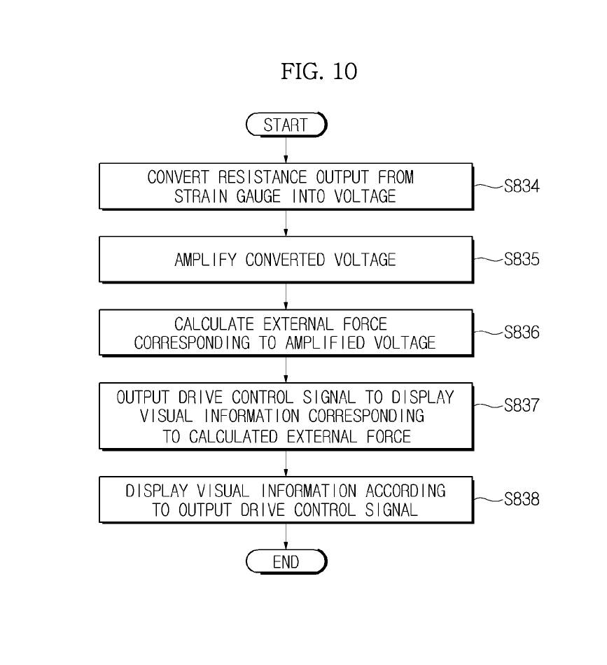

In an embodiment, as exemplarily shown in FIG. 10, the resistance output from the strain gauge 220 is converted into an electric voltage signal to be measured (S834). The converted voltage is amplified to a measurable magnitude (S835). External force corresponding to the amplified voltage is calculated (S836). Thereafter, when a drive control signal to display visual information corresponding to the calculated external force is output (S837), the LED display 233B displays the visual information in response to the output drive control signal (S838).

A conversion of the resistance output from the strain gauge 220 into the voltage may be performed using the Wheatstone bridge 231c as described above, and amplification of the converted voltage may be performed using the signal amplifier 231d.

In addition, calculation of the external force corresponding to the voltage amplified by the signal amplifier 231d may be performed via the drive controller 231e as exemplarily shown in FIG. 5. Although calculation of the external force may be performed by substituting the voltage into a preset function by way of example, the calculation method is not in any way limited thereto.

In addition, the drive controller 231e may first calculate external force, and thereafter read the drive control signal for display of visual information corresponding to the calculated external force from the storage unit (not shown) to output the drive control signal to the LED display device 233B. The ‘drive control signal’ may be a signal to control whether or not current is applied to the respective LEDs included in the LED display device 233B, but is not in any way limited thereto.

That is, in an embodiment, the resistance output from the strain gauge 220, i.e. an invisible electric signal is represented as visual information. For example, the galvanometer 233A visually represents the resistance output from the strain gauge 220 via movement of the pointer, whereas the LED display device 233B may visually represent letters including numbers or pictures including specific figures. A ‘pointer movement’, ‘letters’, ‘figures’, ‘pictures’, etc. may correspond to ‘visual information’.

Next, an image with regard to the visual information displayed on the galvanometer 233A or the LED display device 233B is acquired (S840), and external force information corresponding to the visual information is calculated using the acquired image (S850). The calculated external force information is transmitted to the master device 100 (S860).

Acquisition of the image with regard to the visual information may be performed using the image capture unit 240. The image capture unit 240 may include a CCD camera and an endoscope camera, but is not in any way limited thereto, and any other device may be used so long as it may form an image.

After acquisition of the image with regard to the visual information displayed via the image capture unit 240 or the LED display device 233B, the image capture unit 240 may transmit the acquired image to the controller 250.

The controller 250 may extract visual information from the image by performing the known image processing on the image transmitted from the image capture unit 240, and may calculate external force information corresponding to the extracted visual information. For example, the controller 250 may extract visual information, such as the direction of the pointer of the galvanometer 233A, a number of a scale indicated by the pointer, etc. Alternatively, the controller 250 may extract visual information, such as letters, figures, pictures, etc., displayed on the LED display device 233B, and calculate external force information corresponding to the visual information.

In addition, the controller 250 may transmit the calculated external force information to the master device 100 using the communication unit (not shown). The master device 100 may feed back the transmitted external force information to the input unit 112 to allow the operator to sense the external force applied to the surgical tools 206 and 208.

Although an embodiment in which the image with regard to the visual information acquired by the image capture unit 240 is transmitted to the controller 250 of the slave device 200 has been described above, this is given by way of example, and the image with regard to the visual information acquired by the image capture unit 240 may be transmitted to the controller (not shown) of the master device 100 via a network. The controller (not shown) of the master device 100 may extract visual information by performing image processing on the visual information transmitted from the image capture unit 240, and convert the extracted visual information into corresponding external force information and feed back the converted information to the input unit 112.

Processes, functions, methods, and/or software in apparatuses described herein may be recorded, stored, or fixed in one or more non-transitory computer-readable storage media (computer readable recording medium) that includes program instructions (computer readable instructions) to be implemented by a computer to cause one or more processors to execute or perform the program instructions. The media may also include, alone or in combination with the program instructions, data files, data structures, and the like. The media and program instructions may be those specially designed and constructed, or they may be of the kind well-known and available to those having skill in the computer software arts. Examples of non-transitory computer-readable storage media include magnetic media, such as hard disks, floppy disks, and magnetic tape; optical media such as CD ROM disks and DVDs; magneto-optical media, such as optical disks; and hardware devices that are specially configured to store and perform program instructions, such as read-only memory (ROM), random access memory (RAM), flash memory, and the like. Examples of program instructions include machine code, such as produced by a compiler, and files containing higher level code that may be executed by the computer using an interpreter. The program instructions may be executed by one or more processors. The described hardware devices may be configured to act as one or more software modules that are recorded, stored, or fixed in one or more computer-readable storage media, in order to perform the operations and methods described above, or vice versa. In addition, a non-transitory computer-readable storage medium may be distributed among computer systems connected through a network and computer-readable codes or program instructions may be stored and executed in a decentralized manner. In addition, the computer-readable storage media may also be embodied in at least one application specific integrated circuit (ASIC) or Field Programmable Gate Array (FPGA).

Although embodiments of have been shown and described, it would be appreciated by those skilled in the art that changes may be made in these embodiments without departing from the principles and spirit of the disclosure, the scope of which is defined in the claims and their equivalents.

What is claimed is:

1. A surgical robot comprising a slave device that includes a robot arm, to which a surgical instrument provided with a surgical tool is coupled, and a master device that controls operation of the slave device,
wherein the slave device includes:
an external force measurement unit that includes a force sensor attached to the surgical instrument to measure external force applied to the surgical tool, and a visual information display unit connected to the force sensor to receive the measured external force from the force sensor and display visual information corresponding to the received external force;
an image capture unit that acquires an image corresponding to the visual information displayed on the visual information display unit; and
a controller that extracts the visual information from the acquired image from the image capture unit using image processing, and converts the extracted visual information into corresponding external force information.

2. The surgical robot according to claim 1, wherein the force sensor includes a strain gauge.

3. The surgical robot according to claim 2, wherein the visual information display unit includes:
a visual information display device that displays the visual information; and
a signal processor that converts resistance output from the strain gauge into a signal that may be recognized by the visual information display device, and transmits the converted signal to the visual information display device.

4. The surgical robot according to claim 3, wherein the visual information display device includes a galvanometer.

5. The surgical robot according to claim 4, wherein the signal processor includes:
a Wheatstone bridge that converts the resistance output from the strain gauge into a voltage and outputs the voltage; and
a signal amplifier that amplifies the voltage output from the Wheatstone bridge and outputs the amplified voltage.

6. The surgical robot according to claim 3, wherein the visual information display device includes a Light Emitting Diode (LED) display device.

7. The surgical robot according to claim 6, wherein the signal processor includes:
a Wheatstone bridge that converts the resistance output from the strain gauge into a voltage and outputs the voltage;
a signal amplifier that amplifies the voltage output from the Wheatstone bridge and outputs the amplified voltage; and
a drive controller that receives the amplified voltage output from the signal amplifier, calculates external force corresponding to the voltage, and outputs a drive control signal for display of visual information corresponding to the calculated external force to the LED display device so as to assist the LED display device in displaying the visual information.

8. The surgical robot according to claim 1, wherein the image capture unit includes a Charge Coupled Device (CCD) camera and an endoscope camera.

9. The surgical robot according to claim 1, wherein the slave device further includes a communication unit, and
wherein the controller transmits the external force information to the master device through the communication unit.

10. The surgical robot according to claim 1, wherein the slave device further includes a robot arm to which the image capture unit is coupled.

11. A control method for a surgical robot comprising a slave device that includes a robot arm, to which a surgical instrument provided with a surgical tool is coupled, and a master device that controls operation of the slave device, the method comprising:
measuring external force applied to the surgical tool;
displaying visual information corresponding to the measured external force;
acquiring an image with corresponding to the displayed visual information; and
calculating external force information corresponding to the visual information using the acquired image.

12. The control method according to claim 11, wherein measurement of the external force applied to the surgical tool is performed using a force sensor attached to an end of the surgical instrument provided with the surgical tool.

13. The control method according to claim 12, wherein the force sensor includes a strain gauge.

14. The control method according to claim 13, wherein display of the visual information corresponding to the external force includes:
converting resistance output from the strain gauge into a voltage;
amplifying the converted voltage; and
measuring and displaying the amplified voltage.

15. The control method according to claim 14, wherein measurement and display of the voltage are performed using a galvanometer.

16. The control method according to claim 14, wherein conversion of the resistance output from the strain gauge into the voltage is performed using a Wheatstone bridge.

17. The control method according to claim 13, wherein display of the visual information corresponding to the external force includes:
converting the resistance output from the strain gauge into a voltage;
amplifying the converted voltage;
calculating external force corresponding to the amplified voltage;
outputting a drive control signal for display of visual information corresponding to the calculated external force; and
displaying the visual information according to the output drive control signal.

18. The control method according to claim 17, wherein display of the visual information is performed using a Light Emitting Diode (LED) display device.

19. The control method according to claim 17, wherein conversion of the resistance output from the strain gauge into the voltage is performed using a Wheatstone bridge.

20. The control method according to claim 11, wherein acquisition of the image with corresponding to the displayed visual information is performed using a Charge Coupled Device (CCD) camera and an endoscope camera.
